(19) United States
(12) Patent Application Publication
(10) Pub. No.: US 2014/0107453 A1
(43) Pub. Date: Apr. 17, 2014

(54) REAL-TIME SIGNAL COMPARISON TO GUIDE ABLATION CATHETER TO THE TARGET LOCATION

(71) Applicant: Boston Scientific Scimed Inc., Maple Grove, MN (US)

(72) Inventors: Barun Maskara, Blaine, MN (US); Pramodsingh H. Thakur, Woodbury, MN (US); Shantha Arcot-Krishnamurthy, Renton, WA (US); Shibaji Shome, Arden Hills, MN (US); Sunipa Saha, Shoreview, MN (US); Allan C. Shuros, St. Paul, MN (US)

(73) Assignee: Boston Scientific Scimed Inc., Maple Grove, MN (US)

(21) Appl. No.: 14/053,573

(22) Filed: Oct. 14, 2013

Related U.S. Application Data
(60) Provisional application No. 61/713,787, filed on Oct. 15, 2012.

(51) Int. Cl. A61B 5/06; A61B 5/00; A61B 18/14; A61B 5/042

(52) U.S. Cl. CPC A61B 5/068; A61B 5/0422; A61B 5/4836; A61B 18/1492; A61B 5/746; A61B 5/742; A61B 5/6858; A61B 5/066
USPC 600/374

(57) ABSTRACT

A catheter system includes a plurality of mapping electrodes, an electrode movable relative to the plurality of mapping electrodes, and a guidance system coupled to the plurality of mapping electrodes and the ablation electrode. The guidance system is configured to receive signals associated with intrinsic cardiac activity sensed by the plurality of mapping electrodes and the movable electrode, and to correlate in real-time the intrinsic cardiac activity sensed by the movable electrode with the intrinsic cardiac activity sensed by the plurality of mapping electrodes based on the signals received by the plurality of mapping electrodes and movable electrode to determine a location of the movable electrode with respect to the plurality of mapping electrodes.

### CROSS-REFERENCE TO RELATED APPLICATION

This application claims the benefit of U.S. Provisional Application 61/713,787, filed Oct. 15, 2012, which is incorporated herein by reference in its entirety.

### TECHNICAL FIELD

The present disclosure relates to mapping systems. More particularly, the present disclosure relates to a mapping system configured to cross-correlate intrinsic physiological signals sensed by a mapping catheter to intrinsic physiological signals sensed by an ablation catheter.

### BACKGROUND

Physicians make use of catheters in medical procedures to gain access into interior regions of the body for diagnostic and therapeutic purposes. It is important for the physician to be able to precisely position the catheter within the body to gain contact with a desired tissue location. During these procedures, a physician steers the catheter through a main vein or artery into the interior region of the heart that is to be treated. The physician then further manipulates a steering mechanism to place the electrode carried on the distal tip of the catheter into direct contact with the endocardial tissue. The physician directs energy from the electrode through myocardial tissue either to an indifferent electrode (in a unipolar electrode arrangement) or to an adjacent electrode (in a bi-polar electrode arrangement) to ablate the tissue.

Before ablating heart tissue, physicians often examine the propagation of electrical impulses in heart tissue to locate aberrant conductive pathways and to identify the arrhythmia foci, which are ablated. The techniques used to analyze these pathways and locate foci are commonly called mapping.

### SUMMARY

Disclosed herein are various embodiments of a method for cross-correlating intrinsic physiological signals sensed by a mapping catheter to intrinsic physiological signals sensed by an ablation catheter, as well as cardiac mapping systems employing such methods.

In Example 1, a catheter system includes a plurality of mapping electrodes, an electrode movable relative to the plurality of mapping electrodes, and a guidance system coupled to the plurality of mapping electrodes and the ablation electrode. The guidance system is configured to receive signals associated with intrinsic cardiac activity sensed by the plurality of mapping electrodes and the movable electrode, and to correlate in real-time the intrinsic cardiac activity sensed by the movable electrode with the intrinsic cardiac activity sensed by the plurality of mapping electrodes based on the signals received by the plurality of mapping electrodes and movable electrode to determine a location of the movable electrode with respect to the plurality of mapping electrodes.

In Example 2, the catheter system according to Example 1, wherein the guidance system is configured to generate an alert when the movable electrode is located at a target site.

In Example 3, the catheter system according to Example 1 or Example 2, wherein the guidance system is configured to receive signals associated with a rotor from the plurality of mapping electrodes and the movable electrode.

In Example 4, the catheter system according to any of Examples 1-3, and further comprising a display coupled to the guidance system configured to present a real-time position-identifying output of the movable electrode with respect to the plurality of mapping electrodes.

In Example 5, the catheter system according to any of Examples 1-4, wherein the plurality of mapping electrodes are carried on a basket structure at the end of a mapping catheter.

In Example 6, the catheter system according to any of Examples 1-5, wherein the movable electrode is coupled to a source of ablation energy.

In Example 7, the catheter system according to any of Examples 1-6, wherein the movable electrode is disposed on a movable catheter body.

In Example 8, a method for guiding a movable electrode with respect to a plurality of mapping electrodes includes positioning the movable electrode with respect to the plurality of mapping electrodes, sensing intrinsic cardiac activity with the movable electrode and the plurality of mapping electrodes, and correlating in real-time the intrinsic cardiac activity sensed by the movable electrode with the intrinsic cardiac activity sensed by the plurality of mapping electrodes based on signals associated with the intrinsic cardiac activity received by the plurality of mapping electrodes and movable electrode. The method further includes generating an output based on the signals associated with the intrinsic cardiac activity sensed by the plurality of mapping electrodes and movable electrode, the output providing information that locates the movable electrode relative to the plurality of mapping electrodes.

In Example 9, the method according to Example 8, and further comprising repositioning the movable electrode with respect to the plurality of mapping electrodes, and updating the output based on the intrinsic cardiac activity sensed by the plurality of mapping electrodes and the repositioned movable electrode.

In Example 10, the method according to either Example 8 or Example 9, wherein the sensing step comprises sensing a rotor with the plurality of mapping electrodes.

In Example 11, the method according to any of Examples 8-10, wherein the correlating step comprises deriving a path of the rotor based on the rotor sensed by the plurality of mapping electrodes, identifying the electrodes among the plurality of mapping electrodes that are disposed along the path of the rotor, comparing in real-time the intrinsic cardiac activity sensed by the movable electrode with the rotor path sensed by the plurality of electrodes, and identifying the electrodes disposed along the path of the rotor having a strongest correlation to the intrinsic cardiac activity sensed by the movable electrode.

In Example 12, the method according to any of Examples 8-11, wherein comparing in real-time the intrinsic cardiac activity sensed by the movable electrode with the rotor path sensed by the plurality of electrodes comprises establishing a correlation coefficient for each electrode along the path of the rotor that is a function of the correlation between the rotor path sensed by the electrode and the intrinsic cardiac activity sensed by the movable electrode.

In Example 13, the method according to any of Examples 8-12, wherein the generating step comprises presenting a real-time position-identifying output of the movable electrode with respect to the plurality of mapping electrodes.

In Example 14, the method according to any of Examples 8-13, wherein the plurality of mapping electrodes are carried on a basket structure at the end of a mapping catheter.

In Example 15, a method for guiding a movable electrode with respect to a plurality of mapping electrodes includes positioning the plurality of mapping electrodes proximate to an anatomical structure, sensing intrinsic physiological activity of the anatomical structure with the plurality of mapping electrodes, positioning the movable electrode with respect to the plurality of mapping electrodes, and sensing intrinsic physiological activity of the anatomical structure with the movable electrode. The method also includes correlating in real-time the intrinsic physiological activity sensed by the movable electrode with the intrinsic physiological activity sensed by the plurality of mapping electrodes. The method further includes generating an output based on the intrinsic physiological activity sensed by the plurality of mapping electrodes and movable electrode, the output providing information that locates the movable electrode relative to the plurality of mapping electrodes.

In Example 16, the method according to Example 15, and further comprising repositioning the movable electrode with respect to the plurality of mapping electrodes, and updating the output based on the intrinsic physiological activity sensed by the plurality of mapping electrodes and the repositioned movable electrode.

In Example 17, the method according to Example 15 or Example 16, wherein the sensing step comprises sensing a cardiac rotor with the plurality of mapping electrodes.

In Example 18, the method according to any of Example 15-17, wherein the correlating step comprises deriving a path of the rotor based on the rotor sensed by the plurality of mapping electrodes, identifying the electrodes among the plurality of mapping electrodes that are disposed along the path of the rotor, comparing in real-time the intrinsic physiological activity sensed by the movable electrode with the rotor path sensed by the plurality of electrodes, and identifying the electrodes disposed along the path of the rotor having a strongest correlation to the intrinsic physiological activity sensed by the movable electrode.

In Example 19, the method according to any of Example 15-18, wherein comparing in real-time the intrinsic physiological activity sensed by the movable electrode with the rotor path sensed by the plurality of electrodes comprises establishing a correlation coefficient for each electrode along the path of the rotor that is a function of the correlation between the rotor path sensed by the electrode and the intrinsic physiological activity sensed by the movable electrode.

In Example 20, the method according to any of Example 15-19, wherein the generating step comprises presenting a real-time position-identifying output of the movable electrode with respect to the plurality of mapping electrodes.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an embodiment of a system for accessing a targeted tissue region in the body for diagnostic and therapeutic purposes.

FIG. 2 is a schematic view of an embodiment of a mapping catheter having a basket functional element carrying structure for use in association with the system of FIG. 1.

FIG. 3 is a schematic view of an embodiment of an ablation catheter for use in association with the system of FIG. 1.

FIG. 4 is a schematic side view of an embodiment of the basket functional element including a plurality of mapping electrodes.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

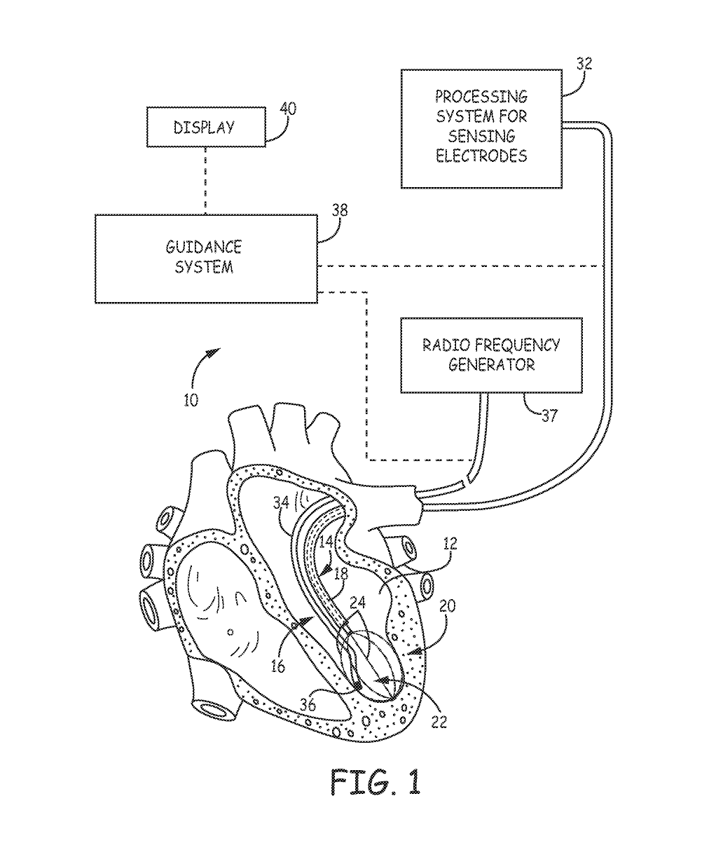

FIG. 1 is a schematic view of a system 10 for accessing a targeted tissue region in the body for diagnostic or therapeutic purposes. FIG. 1 generally shows the system 10 deployed in the left ventricle of the heart. Alternatively, system 10 can be deployed in other regions of the heart, such as the left atrium, right atrium, or right ventricle. While the illustrated embodiment shows the system 10 being used for ablating heart tissue, the system 10 (and the methods described herein) may alternatively be configured for use in other tissue ablation applications, such as procedures for ablating tissue in the prostrate, brain, gall bladder, uterus, and other regions of the body, including in systems that are not necessarily catheter-based.

The system 10 includes a mapping probe 14 and an ablation probe 16. In FIG. 1, each is separately introduced into the selected heart region 12 through a vein or artery (e.g., the femoral vein or artery) through suitable percutaneous access. Alternatively, the mapping probe 14 and ablation probe 16 can be assembled in an integrated structure for simultaneous introduction and deployment in the heart region 12.

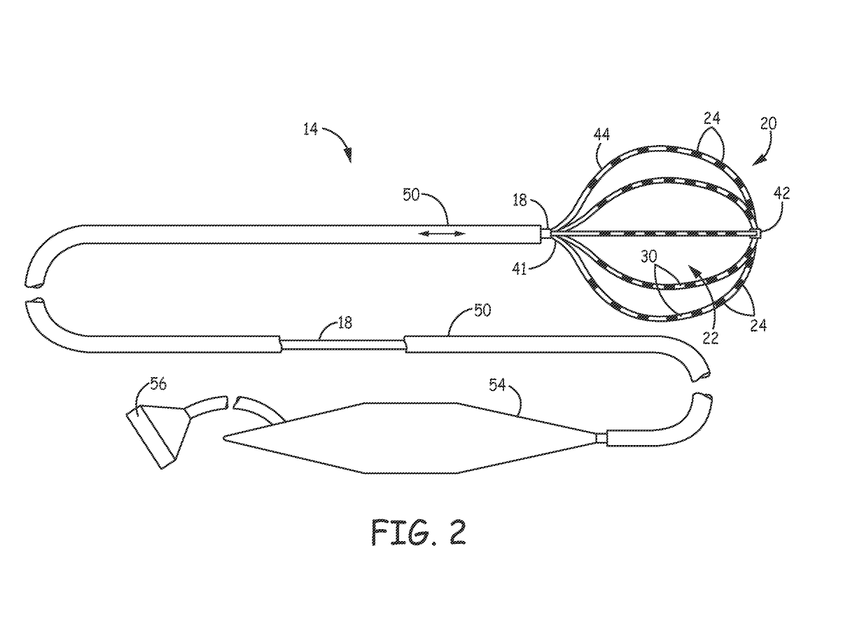

The mapping probe 14 has a flexible catheter body 18. The distal end of the catheter body 18 carries a three-dimensional multiple electrode structure 20. In the illustrated embodiment, the structure 20 takes the form of a basket defining an open interior space 22 (see FIG. 2), although other multiple electrode structures could be used. The multiple electrode structure 20 carries a plurality of electrodes 24 configured to sense intrinsic physiological activity in the anatomical region on which the ablation procedure is to be performed.

The electrodes 24 are electrically coupled to a processing system 32. A signal wire (not shown) is electrically coupled to each electrode 24 on the basket structure 20. The wires extend through the body 18 of the probe 14 and electrically couple the electrodes 24 to the processing system 32 (and the guidance system 34, as will be described later in greater detail). The electrodes 24 sense intrinsic electrical activity in heart tissue. The sensed activity is processed by the processing system 32 to assist the physician in identifying the site or sites within the heart appropriate for ablation.

In some embodiments, the processing system 32 may be configured to measure the intrinsic electrical activity in the heart tissue adjacent to the electrodes 24. For example, in some embodiments, the processing system 32 is configured to detect intrinsic electrical activity associated with a dominant rotor in the anatomical feature being mapped. Studies have shown that dominant rotors have a role in the initiation and maintenance of atrial fibrillation, and ablation of the rotor path and/or rotor core may be effective in terminating the atrial fibrillation. In either situation, the processing system 32 processes the sensed information to derive the location of a site appropriate for ablation using the ablation probe 16.

The ablation probe 16 includes a flexible catheter body 34 that carries one or more ablation electrodes 36. The one or more ablation electrodes 36 are electrically connected to a radio frequency generator 37 that is configured to deliver ablation energy to the one or more ablation electrodes 36. The ablation probe 16 is movable with respect to the anatomical feature to be treated, as well as the structure 20. The ablation probe 16 is positionable between or adjacent to electrodes 24 of the structure 20 as the one or more ablation electrodes 36 are positioned with respect to the tissue to be treated.

A guidance system 38 is electrically coupled to the mapping catheter 14 and the ablation catheter 16. The guidance system 38 collects and processes information regarding the location of the ablation probe 16 within the space 22 defined by the basket structure 20, in term of its position relative to the position of the electrodes 24. The guidance system 38 provides a position-identifying output that aids the physician in guiding the ablation one or ablation electrodes 36 into contact with tissue at the site identified for ablation. The guidance system 38 can process and provide position-specific information in various ways. In some embodiments, the guidance system 38 cross-correlates intrinsic physiological signals sensed by the electrodes 24 on the structure 20 with intrinsic physiological signal sensed by the one or ablation electrodes 36

In the illustrated embodiment, the guidance system 38 includes an output display device 40 (e.g., a CRT, LED display, or a printer). The device 40 presents the position-identifying output in a real-time format most useful to the physician for remotely guiding the ablation electrode 36 within the basket structure 20.

FIG. 2 illustrates an embodiment of the mapping catheter 14 including electrodes 24 at the distal end suitable for use in the system 10 shown in FIG. 1. The mapping catheter 14 has a flexible catheter body 18, the distal end of which carries the three dimensional structure 20 configured to carry the mapping electrodes or sensors 24. The mapping electrodes 24 sense intrinsic electrical activity in the heart tissue, which sensed activity is then processed by the processing system 32 and guidance system 38 to assist the physician in identifying the site or sites having a heart rhythm disorder. This process is commonly referred to as mapping. This information can then be used to determine an appropriate location for applying appropriate therapy (e.g., ablation) to the identified sites, and to navigate the one or more ablation electrodes 36 to the identified sites.

The illustrated three dimensional structure 20 comprises a base member 41 and an end cap 42 between which flexible splines 44 generally extend in a circumferentially spaced relationship. As discussed above, the three dimensional structure 20 takes the form of a basket defining an open interior space 22. In some embodiments, the splines 44 are made of a resilient inert material, such as, e.g., Nitinol metal or silicone rubber, and are connected between the base member 41 and the end cap 42 in a resilient, pretensed condition, to bend and conform to the tissue surface they contact. In the illustrated embodiment, eight splines 44 form the three dimensional structure 20. Additional or fewer splines 44 could be used in other embodiments. As illustrated, each spline 44 carries eight mapping electrodes 24. Additional or fewer mapping electrodes 24 could be disposed on each spline 44 in other embodiments of the three dimensional structure 20. In the illustrated embodiment, the three dimensional structure 20 is relatively small (e.g., 40 mm or less in diameter). In alternative embodiments, the three dimensional structure 20 is larger (e.g., 40 mm in diameter or greater).

A slidable sheath 50 is movable along the major axis of the catheter body 30. Moving the sheath 50 forward (i.e., toward the distal end) causes the sheath 50 to move over the three dimensional structure 20, thereby collapsing the structure 20 into a compact, low profile condition suitable for introduction into an interior space, such as, for example, into the heart. In contrast, moving the sheath 50 rearward (i.e., toward the proximal end) frees the three dimensional structure 20, allowing the structure 20 to spring open and assume the pretensed position illustrated in FIG. 2. Further details of embodiments of the three dimensional structure 20 are disclosed in U.S. Pat. No. 5,647,870, entitled “Multiple Electrode Support Structures,” which is hereby incorporated by reference in its entirety.

A signal wire (not shown) is electrically coupled to each mapping electrode 26. The wires extend through the body 30 of the mapping catheter 20 into a handle 54, in which they are coupled to an external connector 56, which may be a multiple pin connector. The connector 56 electrically couples the mapping electrodes 24 to the processing system 32 and guidance system 38. Further details on mapping systems and methods for processing signal generated by the mapping catheter are discussed in U.S. Pat. No. 6,070,094, entitled “Systems and Methods for Guiding Movable Electrode Elements within Multiple-Electrode Structure,” U.S. Pat. No. 6,233,491, entitled “Cardiac Mapping and Ablation Systems,” and U.S. Pat. No. 6,735,465, entitled “Systems and Processes for Refining a Registered Map of a Body Cavity,” the disclosures of which are incorporated herein by reference.

It is noted that other multi-electrode structures could be deployed on the distal end. It is further noted that the multiple mapping electrodes 24 may be disposed on more than one structure rather than, for example, the single mapping catheter 14 illustrated in FIG. 2. For example, if mapping within the left atrium with multiple mapping structures, an arrangement comprising a coronary sinus catheter carrying multiple mapping electrodes and a basket catheter carrying multiple mapping electrodes positioned in the left atrium may be used. As another example, if mapping within the right atrium with multiple mapping structures, an arrangement comprising a decapolar catheter carrying multiple mapping electrodes for positioning in the coronary sinus, and a loop catheter carrying multiple mapping electrodes for positioning around the tricuspid annulus may be used.

Additionally, although the mapping electrodes 24 have been described as being carried by dedicated probes, such as mapping catheter 14, the mapping electrodes can be carried on non-mapping dedicated probes. For example, an ablation catheter (e.g., the ablation catheter 16) can be configured to include one or mapping electrodes 24 disposed on the distal end of the catheter body and coupled to the signal processing system 32 and guidance system 38. As another example, the ablation electrode at the distal end of the ablation catheter may be coupled to the signal processing system 32 and guidance system 38 to also operate as a mapping electrode.

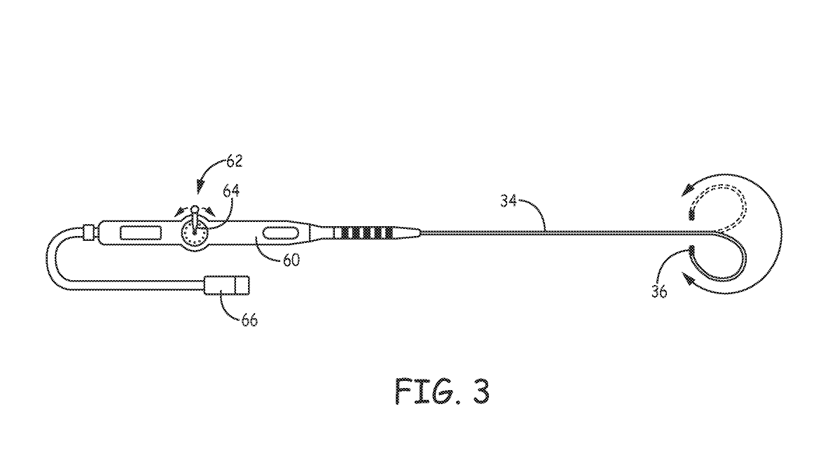

FIG. 3 is a schematic view of an embodiment of the ablation catheter 16 for use in association with the system of FIG. 1. For the sake of illustration, FIG. 1 shows a single ablation electrode 36 carried at the distal tip of the catheter body 34. Other configurations employing multiple ablation electrodes are possible, as described in U.S. Pat. No. 5,582,609, entitled “Systems and Methods for Forming Large Lesions in Body Tissue Using Curvilinear Electrode Elements,” which is hereby incorporated by reference in its entirety.

A handle 60 is attached to the proximal end of the catheter body 34. The handle 60 and catheter body 34 carry a steering mechanism 62 for selectively bending or flexing the catheter body 34 along its length, as the arrows in FIG. 3 show. In the illustrated embodiment, the steering mechanism 62 includes a rotating cam wheel with an external steering lever 64. Movement of the steering lever 64 flexes the distal end of the body 34 to bring the electrode 36 into conforming, intimate contact against the endocardial tissue. One exemplary steering mechanism is shown and described in U.S. Pat. No. 5,254,088, which is hereby incorporated by reference in its entirety.

A wire (not shown) electrically connected to the ablation electrode 36 extends through the catheter body 34 into the handle 60, where it is electrically coupled to an external connector 66. The connector 66 connects the electrode 36 to the radio frequency ablation energy generator 37 and to the guidance system 38 (FIG. 1). In use, the physician places the ablation electrode 36 in contact with heart tissue at the site identified by the mapping probe 14 for ablation. The ablation electrode 36 emits ablating energy to heat and thermally destroy the contacted tissue.

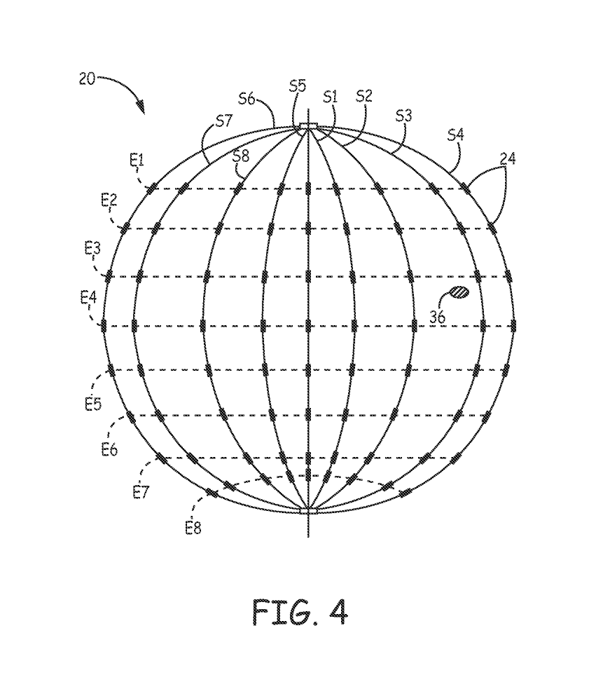

To illustrate the operation of the system 10, FIG. 4 is a schematic side view of an embodiment of the basket structure 20 including a plurality of mapping electrodes 24. In the illustrated embodiment, the basket structure includes 64 mapping electrodes 24. The mapping electrodes 24 are disposed in groups of eight electrodes (labeled E1, E2, E3, E4, E5, E6, E7, and E8) on each of eight splines (labeled S1, S2, S3, S4, S5, S6, S7, and S8). While the sixty-four mapping electrodes 24 are shown disposed on a basket structure 20, the mapping electrodes 24 may alternatively be arranged in different numbers and on different structures.

After the basket structure 20 is positioned adjacent to the anatomical structure to be treated (e.g., left atrium or left ventricle of the heart), the processing system 32 and guidance system 38 receive signals from the electrodes 24 related to intrinsic physiological activity of the anatomical structure. That is, the electrodes 24 measure the electrical impulses intrinsic to the physiology of the anatomical structure.

The ablation electrode 36 of the ablation catheter 16 is moved relative to the basket structure 20 until the ablation electrode 36 is positioned adjacent to or against the anatomical structure to be treated proximate to the basket structure 20. The ablation electrode 36 may pass through the interior space 22 of the basket structure 20 to reach the anatomical structure to be treated, for example. For example, in the embodiment illustrated in FIG. 4, the ablation electrode 36 is disposed against the anatomical structure between the mapping electrodes E3 and E4 on splines S2 and S3. The ablation electrode 36 (or one or more other electrodes on the ablation catheter 16) then senses signals related to the intrinsic physiological activity of the anatomical structure, which are provided to the guidance system 38.

The guidance system 38 then correlates in real-time the intrinsic physiological activity sensed by the ablation electrode 36 (or other electrodes on the ablation catheter 16) with the intrinsic physiological activity sensed by mapping electrodes 24 based on the signals received from the mapping electrodes 24 and the ablation electrode 36. For example, in some embodiments, the guidance system 38 identifies the mapping electrodes 24 that provide the intrinsic physiological activity to be targeted by the ablation procedure. The guidance system then compares, in real-time, the physiological activity sensed by the ablation electrode 36 (or other movable electrodes) with the targeted intrinsic physiological activity sensed by the mapping electrodes 24. In some embodiments, the guidance system 38 establishes a correlation coefficient for each mapping electrode 24 around the targeted physiological activity that is a function of a correlation or similarity between the physiological activity by the electrode and the physiological activity sensed by the ablation electrode 36. The guidance system 38 then identifies the one or more mapping electrodes 24 having the strongest signal correlation to the physiological activity sensed by the ablation electrode 36. For example, in the embodiment illustrated in FIG. 4, the guidance system 38 would identify the signal associated with the intrinsic physiological activity on electrode E3 on the spline S3 as having the strongest correlation to the signal sensed by the ablation electrode 36.

The guidance system 38 may then generate an output based on the correlation between the intrinsic physiological activity sensed by the mapping electrodes 24 with the intrinsic physiological activity sensed by the ablation electrode 36. In some embodiments, the guidance system 38 presents a real-time position-identifying output of the ablation electrode 36 with respect to the mapping electrodes 24. For example, the display 40 associated with the guidance system 38 (FIG. 1) may present a representation of the mapping electrodes 24 and the ablation electrode 36 based on the correlated signals. The display 40 may highlight (e.g., with a different color) the one or more mapping electrodes 24 with the strongest signal correlation to the signals sensed by the ablation electrode 36. The display 40 may also present a representation of the relative positioning of the ablation electrode 36 with respect to the array of mapping electrodes 24, based on the signal correlation described above. The display 40 may further present a representation of the intrinsic physiological activity sensed by the mapping electrodes 24 in conjunction with the representation of the mapping electrodes 24 and ablation electrode 36 to assist the clinician in targeting tissue having a particular physiological activity.

Based on the output generated by the guidance system 38, the clinician may then decide to reposition the ablation electrode 36 with respect to the anatomical structure to be treated. For example, based on the output provided on the display 40, the clinician may determine that the ablation electrode 36 is positioned away from the target treatment site. When the clinician moves the ablation electrode 36, the guidance system 38 is configured to updated, in real-time, the output based on an updated correlation between the intrinsic physiological activity sensed by the mapping electrodes 24 and the ablation electrode 36.

When the ablation electrode 36 is located in the desired position, ablation energy may be delivered to the ablation electrode 36 to ablate the target tissue of the anatomical structure.

In an exemplary embodiment, the mapping electrodes 24 are configured to sense and provide signals related to a rotor in the left atrium of the heart, which are provided to the guidance system 38. The guidance system 38 then derives a path and/or core of the rotor based on the signals sensed by the mapping electrodes 24. Based on the derived path and/or core of the rotor, the guidance system 38 identifies the mapping electrodes 24 that are disposed along the path and/or at the core of the rotor. The ablation electrode 36 is then moved into proximity of the identified rotor, and the intrinsic cardiac activity sensed by the ablation electrode 36 (or other electrodes on the ablation catheter 16) is correlated with the signals associated with the rotor, such as using the process described above. Based on this correlation, the guidance system 38 provides an output that indicates whether the ablation electrode 36 is in proximity to the mapping electrodes 24 along the path and/or core of the rotor (e.g, a representation of the mapping electrodes 24 and ablation electrode 36 on the display 40). The ablation electrode 36 can then be repositioned, with the output from the guidance system 38 updating in real-time, until the ablation electrode 36 is disposed in the desired position with respect to the rotor path or core. When in the desired position, ablation energy can be delivered to the ablation electrode 36 to ablate the rotor path or core.

The sensing of a rotor path and/or core with the mapping electrodes 24 and ablation electrode 36 to determine an ablation location is merely exemplary. The guidance system 38 may also be configured to detect other intrinsic physiological responses with repeated activation patterns, such as focal firing, to correlate the location of the ablation electrode 36 with respect to the mapping electrodes 24.

The system 10 described relates to locating a movable electrode 38 with respect to a plurality of mapping electrodes 24 based on correlation of signals associated with intrinsic physiological activity sensed by the movable and mapping electrodes. However, the system 10 described may be configured to determine the position of the movable electrode 38 with respect to the mapping electrodes 24 using alternative or additional means. For example, the system 10 may include means for locating the movable electrode 38 with respect to the mapping electrodes 24 using any of impedance, rate and regularity similarity, ultrasound, magnetic field triangulation technologies.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter system comprising:
a plurality of mapping electrodes;
an electrode movable relative to the plurality of mapping electrodes;
a guidance system coupled to the plurality of mapping electrodes and the ablation electrode, the guidance system configured to receive signals associated with intrinsic cardiac activity sensed by the plurality of mapping electrodes and the movable electrode, the guidance system further configured to correlate in real-time the intrinsic cardiac activity sensed by the movable electrode with the intrinsic cardiac activity sensed by the plurality of mapping electrodes based on the signals received by the plurality of mapping electrodes and movable electrode to determine a location of the movable electrode with respect to the plurality of mapping electrodes.

2. The catheter system of claim 1, wherein the guidance system is configured to generate an alert when the movable electrode is located at a target site.

3. The catheter system of claim 1, wherein the guidance system is configured to receive signals associated with a rotor from the plurality of mapping electrodes and the movable electrode.

4. The catheter system of claim 1, and further comprising:
a display coupled to the guidance system configured to present a real-time position-identifying output of the movable electrode with respect to the plurality of mapping electrodes.

5. The catheter system of claim 1, wherein the plurality of mapping electrodes are carried on a basket structure at the end of a mapping catheter.

6. The catheter system of claim 1, wherein the movable electrode is coupled to a source of ablation energy.

7. The catheter system of claim 1, wherein the movable electrode is disposed on a movable catheter body.

8. A method for guiding a movable electrode with respect to a plurality of mapping electrodes, the method comprising:
positioning the movable electrode with respect to the plurality of mapping electrodes;
sensing intrinsic cardiac activity with the movable electrode and the plurality of mapping electrodes;
correlating in real-time the intrinsic cardiac activity sensed by the movable electrode with the intrinsic cardiac activity sensed by the plurality of mapping electrodes based on signals associated with the intrinsic cardiac activity received by the plurality of mapping electrodes and movable electrode; and
generating an output based on the signals associated with the intrinsic cardiac activity sensed by the plurality of mapping electrodes and movable electrode, the output providing information that locates the movable electrode relative to the plurality of mapping electrodes.

9. The method of claim 8, and further comprising:
repositioning the movable electrode with respect to the plurality of mapping electrodes; and
updating the output based on the intrinsic cardiac activity sensed by the plurality of mapping electrodes and the repositioned movable electrode.

10. The method of claim 8, wherein the sensing step comprises:
sensing a rotor with the plurality of mapping electrodes.

11. The method of claim 10, wherein the correlating step comprises:
deriving a path of the rotor based on the rotor sensed by the plurality of mapping electrodes;
identifying the electrodes among the plurality of mapping electrodes that are disposed along the path of the rotor; and
comparing in real-time the intrinsic cardiac activity sensed by the movable electrode with the rotor path sensed by the plurality of electrodes; and
identifying the electrodes disposed along the path of the rotor having a strongest correlation to the intrinsic cardiac activity sensed by the movable electrode.

12. The method of claim 11, wherein comparing in real-time the intrinsic cardiac activity sensed by the movable electrode with the rotor path sensed by the plurality of electrodes comprises:
establishing a correlation coefficient for each electrode along the path of the rotor that is a function of a correlation between the rotor path sensed by the electrode and the intrinsic cardiac activity sensed by the movable electrode.

13. The method of claim 8, wherein the generating step comprises:
presenting a real-time position-identifying output of the movable electrode with respect to the plurality of mapping electrodes.

14. The method of claim 8, wherein the plurality of mapping electrodes are carried on a basket structure at the end of a mapping catheter.

15. A method for guiding a movable electrode with respect to a plurality of mapping electrodes, the method comprising:
positioning the plurality of mapping electrodes proximate to an anatomical structure;
sensing intrinsic physiological activity of the anatomical structure with the plurality of mapping electrodes;
positioning the movable electrode with respect to the plurality of mapping electrodes;
sensing intrinsic physiological activity of the anatomical structure with the movable electrode;
correlating in real-time the intrinsic physiological activity sensed by the movable electrode with the intrinsic physiological activity sensed by the plurality of mapping electrodes; and
generating an output based on the intrinsic physiological activity sensed by the plurality of mapping electrodes and movable electrode, the output providing information that locates the movable electrode relative to the plurality of mapping electrodes.

16. The method of claim 15, and further comprising:
repositioning the movable electrode with respect to the plurality of mapping electrodes; and
updating the output based on the intrinsic physiological activity sensed by the plurality of mapping electrodes and the repositioned movable electrode.

17. The method of claim 15, wherein the sensing step comprises:
sensing a cardiac rotor with the plurality of mapping electrodes.

18. The method of claim 17, wherein the correlating step comprises:
deriving a path of the rotor based on the rotor sensed by the plurality of mapping electrodes;
identifying the electrodes among the plurality of mapping electrodes that are disposed along the path of the rotor; and
comparing in real-time the intrinsic physiological activity sensed by the movable electrode with the rotor path sensed by the plurality of electrodes; and
identifying the electrodes disposed along the path of the rotor having a strongest correlation to the intrinsic physiological activity sensed by the movable electrode.

19. The method of claim 18, wherein comparing in real-time the intrinsic physiological activity sensed by the movable electrode with the rotor path sensed by the plurality of electrodes comprises:
establishing a correlation coefficient for each electrode along the path of the rotor that is a function of a correlation between the rotor path sensed by the electrode and the intrinsic physiological activity sensed by the movable electrode.

20. The method of claim 15, wherein the generating step comprises:
presenting a real-time position-identifying output of the movable electrode with respect to the plurality of mapping electrodes.
